(19) United States
(12) Patent Application Publication
(10) Pub. No.: US 2025/0288653 A1
(43) Pub. Date: Sep. 18, 2025

(54) ARTIFICIAL BLOOD SUBSTITUTES

(71) Applicant: Thomas Ming Swi CHANG, Montreal (CA)

(72) Inventors: Thomas Ming Swi CHANG, Montreal (CA); Muntasirul HOQ, Montreal (CA)

(21) Appl. No.: 19/066,710

(22) Filed: Feb. 28, 2025

(30) Foreign Application Priority Data
Mar. 15, 2024 (US) 63565735

(51) Int. Cl. A61K 38/51; A61K 38/44; C12N 9/02; C12N 9/08; C12N 9/88

(52) U.S. Cl. CPC A61K 38/51; A61K 38/446; C12N 9/0065; C12N 9/0089; C12N 9/88; C12Y 111/01006; C12Y 115/01001; C12Y 402/01001

(57) ABSTRACT

The present document describes artificial blood substitutes and method of using same. Particularly, the artificial blood substitutes comprising neoenzymes (synthetic enzymes) instead of natural enzymes.

### BACKGROUND

### (a) Field

The subject matter disclosed generally relates to artificial blood substitutes and method of using same. Particularly, the artificial blood substitutes comprising neoenzymes (synthetic enzymes) instead of natural enzymes.

### (b) Related Prior Art

Red blood cells (RBCs) have three major functions: (1) oxygen transport, (2) carbon dioxide transport, and (3) antioxidant properties. Dr. Chang and others prepared different types of nanobiotechnology based blood substitutes (Editor in Chief Chang, coeditors: Bulow, Jahr, Sakai & Yang (all past or present presidents of International Symposia of Blood Substitutes) (2021) A multiauthor book on “Nanobiotheraeutic basis for blood substitutes, World Scientific Publisher/Imperial College (2021). Open Access available to all at: https://www.worldscientific.com/doi/epdf/10.1142/12054. One of these is based on polyhemoglobin using diacid (T. M. S. Chang, Science 1964, 146:524-525) or Glutaraldehyde (T. M. S. Chang, Biochem Biophys Res Common 1971, 44 (6): 1531-153).

There was no initial interest to develop these blood substitutes until 1987 following the major concern regarding HIV in donor blood. This led to “catch up” development of blood substitutes. However, the urgency was such that emphasis was on simplified hemoglobin-based oxygen carriers (HBOC) without the other 2 important functions of red blood cells. One of the more successful one is based on Chang's glutaraldehyde principle. Biopure has produced a glutaraldehyde-crosslinked polyhemoglobin that has undergone extensive clinical trials. South Africa and Russia have approved the routine use of this polyhemoglobin and this has been in use for a number of years (Jahr J S, et al. (2008) J Trauma 64:1484-97). However, it only has only one of the 3 functions of red blood cells, the risk/benefit ratio of polyhemoglobin (polyHb) has been shown in certain areas of the world where the risk of HIV in donor blood is high. In other area of the world where HIV in donor blood is no longer a major problem, the risk/benefit is different.

In severe cases of acute and sustained ischemic conditions, such as those involving ischemia reperfusion (as in myocardial infarction, stroke or organs for transplantation etc), unlike red blood cells, PolyHb is a solution and can more easily reperfuse blocked vessels. However, this would result in the generation of oxygen radicals causing tissue damage. Dr. Chang and his team prepared a nanobiotechnology based PolyHb-Superoxide Dismutase-Catalase (HBOCs with antioxidant properties). Unlike PolyHb, this did not cause ischemia reperfusion injury in rat intestine. Polyhemoglobin being in solution can be utilized in certain conditions, such as myocardial infarction and stroke, as it is more likely to be able to perfuse partially obstructed vessels in those conditions. In a rat hemorrhagic shock-stroke model, Dr. Chang and his team found that after 60 minutes of ischemia, reperfusion using polyHb significantly increased the breakdown of the blood-brain barrier and the development of edema in the brain. But, polyHb-SOD-CAT did not cause these adverse effects.

The major concern related to polyHb is the observation in clinical trials in hemorrhagic shock of a very slight increase in nonfatal myocardial infarction. Despite the fact that red blood cells transport both carbon dioxide and oxygen, the worldwide research on blood substitutes still focuses on the transport of oxygen and removal of oxygen radicals. A study conducted in an animal model in Norway revealed that the degree of tissue PCO₂ elevation is related to the degree of myocardial ischemia.

Thus, Chang's group has developed a nanobiotechnological preparation that can transport both carbon dioxide and oxygen and remove oxygen radicals. It is composed of polyhemoglobin-catalase-superoxide dismutase-carbonic anhydrase (PolyHb-SOD-CAT-CA). Their nanobiotechnology method allows enzyme concentration to be up to 6 times that of red blood cells. In a rat model of hemorrhagic shock, they found that the polyHb-SOD-CAT-CA was more effective than red blood cells at reducing tissue PCO2 levels and protect the heart. This 3rd generation blood substitute containing natural enzymes (Superoxide Dismutase, Catalase and Carbonic Anhydrase) now has all the 3 major functions of red blood cells. However, natural enzymes are difficult to obtain in large amount they are also known for their low stability at body or room temperature. Moreover, the preparation of the complex (PolyHb-SOD-CAT-CA) requires very careful handling to avoid immunological problems.

There is a need for an artificial blood substitute containing neoenzymes (synthetic enzymes) instead of natural enzymes with increased stability of the complex.

### SUMMARY

According to an embodiment, there is provided an artificial blood substitute containing neoenzymes (synthetic enzymes) instead of natural enzymes. This drastically increases the stability of the complex and reduce the cost and complexity and potential immunogenicity of the complex formation as the neoenzymes (neoSOD, neoCAT and neoCA) are much cheaper and more stable than their natural counterparts.

According to another embodiment, there is provided a synthetic enzymatic activity solution comprising at least one compound selected from the group consisting of:
a Neo-Carbonic Anhydrase (neoCA) compound having Carbonic Anhydrase activity chosen from the group consisting of ZnHisGly, (Zinc Histidine Glycerol);
a Neo-Superoxide Dismutase (neoSOD) compound having Superoxide Dismutase activity chosen from the group consisting of MnTmPyP, or Mn(III)tetrakis(1-methyl-4-pyridyl)porphyrin;
a Neo-Catalase (neoCAT) compound having Catalase activity chosen from the group consisting of EUK-134, or 2,2′-[1,2-Ethanediylbis(nitrilomethylidyne)]bis[6-methoxy-phenol manganese complex; Chloro[[2,2′-[1,2-ethanediylbis(nitrilomethylidyne)]bis[6-methoxyphenolato]](2-)-N2,N2′,O1,O1′]-manganese);
a combination thereof;
wherein the at least one compound is in a PBS solution (pH-7.4), saline, Ringer Lactate, cell culture media, organ preservation media, distilled water dH2O or other pharmaceutically acceptable buffer and is adapted to transport carbon dioxide and/or remove oxygen radicals.

According to an embodiment, there is provided an artificial blood substitute solution comprising:
the synthetic enzymatic activity solution of the present invention having a combination of up to 3 compounds; and
an oxygen carrier selected from the group consisting of: PolyHb, other modified hemoglobin (examples include but not limited to other types of polyhemoglobin, conjugated hemoglobin including PEG-hemoglobin, intramolecularly crosslinked hemoglobin, recombinant hemoglobin, bioengineered hemoglobin, nanoencapsulated hemoglobin with PEG-lipid membrane or PEG-PLA membrane or other membranes), perfluorochemicals, synthetic heme, oxygen saturated solution; fluid for cell and organ preservation used in organs and cell preservation for transplantation or for biotechnological production of stem cells and other cells;
wherein the artificial blood substitute solution is adapted to transport both carbon dioxide and oxygen and to remove oxygen radicals.

The ratio of oxygen carrier to the neoCA, neoSOD and neoCAT in the artificial blood substitute solution is oxygen carrier from 0 to 10 g and neoenzymes from 1 mg to 180 mg.

The synthetic enzymatic activity solution preferably consists of the Neo-Carbonic Anhydrase (neoCA) compound having Carbonic Anhydrase activity, the Neo-Superoxide Dismutase (neoSOD) compound having Superoxide Dismutase activity and the Neo-Catalase (neoCAT) compound having Catalase activity.

According to an embodiment, there is provided a method for the treatment of a blood loss, a condition with accumulation of carbon dioxide, an ischemia-reperfusion injury, or a diver's disease in a patient in need thereof, which comprises administering the artificial blood substitute solution of the present invention.

The ischemia-reperfusion injury includes a severe hemorrhagic shock, a stroke, a myocardial infarction, or combinations thereof.

According to an embodiment, there is provided a method for removing carbon dioxide from a fluid, which comprises treating the fluid to the synthetic enzymatic activity solution of the present invention for a time sufficient to remove the carbon dioxide therefrom.

The fluid is chosen from blood and air and blood may be in a dialysis.

When the blood is in an artificial lung, it may be for transporting oxygen in a patient but also with need to remove the accumulated carbon dioxide (for example severe COVID patients).

According to an embodiment, there is provided a method for preservation of organs or cells for transplantation or for preserving cells like stem cells and other cells for industrial production and storage, which comprises treating the organs or the cells with the artificial blood substitute solution of the present invention.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

FIG. 1 illustrates preliminary viability assay of human hepatocytes in a l/R shock in vitro model treated with different solutions also as a feasibility study on its use for the preservation and recovery of ischemic cells and organs for transplantation.

FIG. 2 illustrates viability assay of human hepatocytes in a 180 min I/R shock in vitro model treated with different solutions also as a feasibility study on its use for the preservation and recovery of ischemic cells and organs for transplantation.

FIG. 3 illustrates preliminary viability assay of human cardiomyocytes in a 180 min I/R shock in vitro model treated with different solutions also as a feasibility study on its use for the preservation and recovery of ischemic cells and organs for transplantation. Also as an example of its ability to protect the heart from injury in hemorrhagic shock condition since the use of PolyHb alone in hemorrhagic shock patients have resulted in a small number of cardiac side effects.

FIG. 4 illustrates viability assay of human cardiomyocytes in a 180 min I/R shock in vitro model treated with different solutions also as a feasibility study on its use for the preservation and recovery of ischemic cells and organs for transplantation. Also as an example of its ability to protect the heart from injury in hemorrhagic shock condition since the use of PolyHb alone in hemorrhagic shock patients have resulted in a small number of cardiac side effects.

### DETAILED DESCRIPTION

In embodiments there are disclosed that instead of natural protein enzymes solution, small chemicals with CAT, SOD and/or CA enzyme activities in solution are used to create an enzymatic activity solution.

This enzymatic activity solution can be added without crosslinking to any oxygen carriers such as, without limitation, PolyHb, other modified Hb, (examples include but not limited to other types of polyhemoglobin with other crosslink agents, conjugated hemoglobin including PEG-hemoglobin, intramolecularly crosslinked hemoglobins, recombinant hemoglobins, bioengineered hemoglobins, nanoencapsulated hemoglobin with PEG-lipid membrane or PEG-PLA membrane or other membranes and others), perfluorochemicals, synthetic heme, oxygen saturated solution; and other oxygen carriers, to transport both carbon dioxide and oxygen and to remove oxygen radicals, fluid for cell and organ preservation used in organs and cell preservation for transplantation or for biotechnological production of stem cells and other cells.

Small chemicals with enzyme activities are not immunogenic and thus can be used as a free solution. Replacing the natural enzymes with chemicals with enzyme activities drastically increased the stability of the entire product as is seen from the results. Even after being kept at room temperature and at 37° C. for 2 months, the enzymatic activities of the synthetic enzymes are the same as day 1 (almost 100% activity).

Preparation of Chemicals with Cat, Sod and Ca Enzyme Activities
neoCARBONIC ANHYDRASE (NeoCA):

Neo-Carbonic Anhydrase (neoCA) was synthesized following the protocol established earlier (Zhibo Zhang et al., Separation and Purification Technology. Volume 276. 2021. 119446. ISSN 1383-5866) Briefly,
1. 0.51 g of Zinc Chloride and 1.72 g of L-Histidine was taken into a beaker.
2. 4.8 ml of Glycerol was added to it.
3. This was then stirred at 70 C until homogenous liquid phase formed (around half an hour). The mixture turned light yellow in colour.
4. The mixture was then dried in a vacuum oven at 60 C for 2 hours, which then results in the neoCA (or referred to as ZnHisGly or Zinc Histidine Glycerol); Zn-based deep eutectic solvent; Mimetic Carbonic Anhydrase.

### Neo-SUPEROXIDE DISMUTASE (NeoSOD):

SOD catalyzes the dismutation of superoxide radicals to molecular oxygen and hydrogen peroxide, providing cellular defense against reactive oxygen species. The neoSOD model used for this study was MnTmPyP or Mn(III)tetrakis(1-methyl-4-pyridyl)porphyrin (bought from Sigma Aldrich Canada (Faulkner, K. M. et al. (1994)). The Journal of biological chemistry, 269 (38), 23471-23476). It is a cell permeable SOD mimic that has been used in many studies but never been reported to be used with a modified Hemoglobin blood substitute as an antioxidating agent.

### Neo-CATALASE (NeoCAT):

Catalase catalyzes the decomposition of hydrogen peroxide to water and oxygen. It protects the cell from oxidative damage by reactive oxygen species. EUK-134 or 2,2′-[1,2-Ethanediylbis(nitrilomethylidyne)]bis[6-methoxy-phenolmanganese complex; Chloro[[2,2′-[1,2-ethanediylbis(nitrilomethylidyne)]bis[6-methoxyphenolato](2-)-N2,N2′,O1,O1′]-manganese is a commercially available CAT mimic that exhibits potent antioxidant activities (bought from Sigma Aldrich Canada (Ri, MH. et al., J Mol Model 28, 168 (2022). https://doi.org/10.1007/s00894-022-05129-4)). It has been used in many studies but never been reported to be used with a modified Hemoglobin blood substitute as an antioxidating agent.

The present invention will be more readily understood by referring to the following examples which are given to illustrate the invention rather than to limit its scope.

### Example 1

### Hepatoprotective Effects of Poly-Hemoglobin and Neoenzymes in Solution on an In Vitro Ischemic/Reperfusion (I/R) Model of Human Hepatocytes

This is also a feasibility study on its use for the preservation and recovery of ischemic cells and organs for transplantation. Organs and cells for transplant are usually ischemic during the period they are removed from the diseased patients. The above fluid supplies the needed oxygen and removes the accumulated CO₂ and oxygen radicals and helps the organs and cells to recover before transplantation. This feasibility is shown in our following preliminary hepatocytes study of improved viability.

### Methods:

### Human Hepatocytes Culture:

DMEM media (supplemented with 10% FBS and 1% penicillin/streptomycin) was warmed to 37° C. in a water bath along with CHRM® Medium (thawing media) obtained from Thermofisher (CM7000). The cryopreserved hepatocytes were thawed in 37° C. water bath for <2 min. The vial was then wiped with 70% alcohol in hood and the content was then transferred into the warm CHRM® Medium. It was then centrifuged at 100×g for 10 min. The supernatant was poured off into waste bottle and ˜1 ml of the Plating medium (DMEM with the above supplementation) per 1×10\hat{ }6 total cells i.e., 8 ml for 8×10\hat{ }6 cells was added and mixed gently. Cells were then counted and seeded at a density of 2.4×10\hat{ }5 cells/ml on collagen coated 6 well plates. The plates were marked according to the different groups of study, which were:
1. Control hepatocytes
2. Hepatocytes treated with 20 ul RL (Ringer's Lactate salt solution) after Ischemic shock
3. Hepatocytes treated with 20 ul RL+0.626 mmol/L PolyHb after Ischemic shock
4. Hepatocytes treated with 20 ul RL+0.626 mmol/L PolyHb+0.05% (w/v) Vitamin C after Ischemic shock
5. Hepatocytes treated with 20 ul RL+55.6 mmol/L neoenzymes in solution (neoSOD+neoCAT+neoCA) after Ischemic shock
6. Hepatocytes treated with 0.626 mmol/L PolyHb+55.6 mmol/L neoenzymes in solution after Ischemic shock

The plates were incubated at 37° C. for 6 hrs. After incubation, the plates were agitated gently to loosen debris and the medium was aspirated and replaced with warm supplemented DMEM medium and kept at incubator for 24 hrs.

### I/R In Vitro Model

Also used as a test for the preservation of ischemic cells and organs for transplantation. Organs and cells for transplant are usually ischemic during the period they are removed from the diseased patients. The above fluid supplies the needed oxygen and removes the accumulated CO₂ and oxygen radicals and helps the organs and cells to recover before transplantation. This feasibility is shown in our preliminary hepatocytes study of improved viability.

The plates were taken out of the incubator and were placed in a shaker kept at 37 C along with a constant flow of Nitrogen gas inside the shaker for creating an ischemic model. The cells were kept in that low oxygen environment for 1 hour. The cells were then taken out of the shaker and the media of the cells were replaced with fresh supplemented DMEM media and the following treatment was given into previously labelled wells:
1. Control hepatocytes got only fresh DMEM media
2. Hepatocytes treated with 20 ul RL and fresh DMEM media
3. Hepatocytes treated with 20 ul RL+0.626 mmol/L PolyHb and fresh DMEM media
4. Hepatocytes treated with 20 ul RL+0.626 mmol/L PolyHb+0.05% (w/v) Vitamin C and fresh DMEM media
5. Hepatocytes treated with 20 ul RL+55.6 mmol/L neoenzymes in solution (neoSOD+neoCAT+neoCA) and fresh DMEM media
6. Hepatocytes treated with 0.626 mmol/L PolyHb+55.6 mmol/L neoenzymes in solution and fresh DMEM media

The plates were then placed back at the incubator for 24 hours.

### Preliminary Viability Assay:

The liquid media was aspirated using pipettes. The cells were then washed twice with 2 ml PBS. 1 ml of 4 mg/ml working solution of collagenase was added onto each well. The plate was gently swirled and keep at RTP for 10 min. 1.5 ml (3:1 of media: collagenase) of DMEM Media (supplemented with FBS) was added to stop collagenase. These were then taken into each separate centrifuge tubes. 1 ml of media was added to the plates and washed and transferred them to the respective tubes. It was repeated 2 more times. The cells were then centrifuged at 1500 rpm for 5 min and the supernatant was then discarded followed by re-suspending the pellet with 5 ml of media. The cells were then counted with trypan blue method using the formula: percentage viability=(total no of cells-total no of cells in blue)/total no of cells×100%.

### Results:

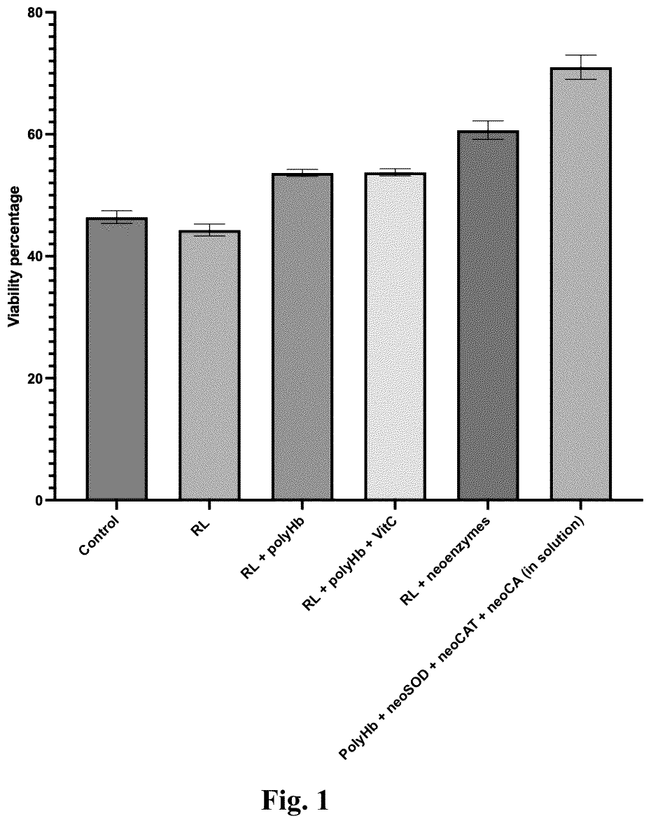

The viability percentage from this preliminary study showed that the polyHb+neoenzymes in solution had a better impact on the cells after a l/R shock as compared to the control and other groups as can be seen in FIG. 1. PolyHb alone could not have a bigger viability percentage, which indicates the production of oxygen radicals that were not removed from the media during the 24 hr incubation. Just neoenzymes had a better viability compared to polyHb, however, that too had limitations as the one with an oxygen carrier i.e., polyHb along with all 3 neoenzymes showed better viability.

### Example 2

### Hepatoprotective Effects of PolyHb+neoCAT+neoSOD+neoCA and PolyHb+neoCAT+neoSOD in Solution on an In Vitro Ischemic/Reperfusion (I/R) Model of Human Hepatocytes

This is also a feasibility study on the use of PolyHb-CAT-SOD-CA for the preservation and recovery of ischemic cells and organs for transplantation. Organs and cells for transplant are usually ischemic during the period they are removed from the diseased patients. The above fluid supplies the needed oxygen and removes the accumulated CO₂ and oxygen radicals and helps the organs and cells to recover before transplantation. This feasibility is shown in our following hepatocytes study of improved viability.

### Methods:

### Human Hepatocytes Culture:

DMEM media (supplemented 1% with 10% FBS and penicillin/streptomycin) was warmed to 37° C. in a water bath along with CHRM® Medium (thawing media) obtained from Thermofisher (CM7000). The cryopreserved hepatocytes were thawed in 37° C. water bath for <2 min. The vial was then wiped with 70% alcohol in hood and the content was then transferred into the warm CHRM® Medium. It was then centrifuged at 100×g for 10 min. The supernatant was poured off into waste bottle and ˜1 ml of the Plating medium (DMEM with the above supplementation) per 1×10\hat{ }6 total cells i.e., 8 ml for 8×10\hat{ }6 cells was added and mixed gently. Cells were then counted and seeded at a density of 2.4×10\hat{ }5 cells/ml on collagen coated 6 well plates. The plates were marked according to the different groups of study, which were:
hepatocytes with no ischemic shock (negative control)

Hepatocytes after Ischemic shock with no treatment (control with no treatment).
Hepatocytes treated with 20 ul RL after Ischemic shock
Hepatocytes treated with 0.626 mmol/L PolyHb after Ischemic shock
Hepatocytes treated with 55.6 mmol/L of three neoenzymes in solution (neoSOD+neoCAT+neoCA) after Ischemic shock
Hepatocytes treated with 0.626 mmol/L PolyHb+55.6 mmol/L of each of three neoenzymes in solution (neoSOD+neoCAT+neoCA) after Ischemic shock
Hepatocytes treated with 0.626 mmol/L PolyHb+55.6 mmol/L of each of two neoenzymes in solution (neoSOD+neoCAT) after Ischemic shock

The plates were incubated at 37° C. for 6 hrs. After incubation, the plates were agitated gently to loosen debris and the medium was aspirated and replaced with warm supplemented DMEM medium and kept at incubator for 24 hrs.

### I/R In Vitro Model

Also used as a test for the preservation of ischemic cells and organs for transplantation. Organs and cells for transplant are usually ischemic during the period they are removed from the diseased patients. The above fluid supplies the needed oxygen and removes the accumulated CO₂ and oxygen radicals and helps the organs and cells to recover before transplantation. This feasibility is shown in our hepatocytes study of improved viability.

The plates were taken out of the incubator and were placed in a shaker kept at 37 C along with a constant flow of Nitrogen gas inside the shaker for creating an ischemic model. The cells were kept in that low oxygen environment for 180 minutes. The cells were then taken out of the shaker and the media of the cells were replaced with fresh supplemented DMEM media and the following treatment was given into previously labelled wells:
Control hepatocytes got only fresh DMEM media
Hepatocytes treated with 20 ul RL
Hepatocytes treated with 0.626 mmol/L PolyHb
Hepatocytes treated with 55.6 mmol/L of each of three neoenzymes in solution (neoSOD+neoCAT+neoCA)
Hepatocytes treated with 0.626 mmol/L PolyHb+55.6 mmol/L of each of three neoenzymes in solution (neoSOD+neoCAT+neoCA)
Hepatocytes treated with 0.626 mmol/L PolyHb+55.6 mmol/L of each of two neoenzymes in solution (neoSOD+neoCAT)

The plates were then placed back at the incubator for 24 hours.

### Viability Assay:

The liquid media was aspirated using pipettes. The cells were then washed twice with 2 ml PBS. 1 ml of 4 mg/ml working solution of collagenase was added onto each well. The plate was gently swirled and keep at RTP for 10 min. 1.5 ml (3:1 of media: collagenase) of DMEM Media (supplemented with FBS) was added to stop collagenase. These were then taken into each separate centrifuge tubes. 1 ml of media was added to the plates and washed and transferred them to the respective tubes. It was repeated 2 more times. The cells were then centrifuged at 1500 rpm for 5 min and the supernatant was then discarded followed by re-suspending the pellet with 5 ml of media. The cells were then counted with trypan blue method using the formula: percentage viability=(total no of cells−total no of cells in blue)/total no of cells×100%.

### Results:

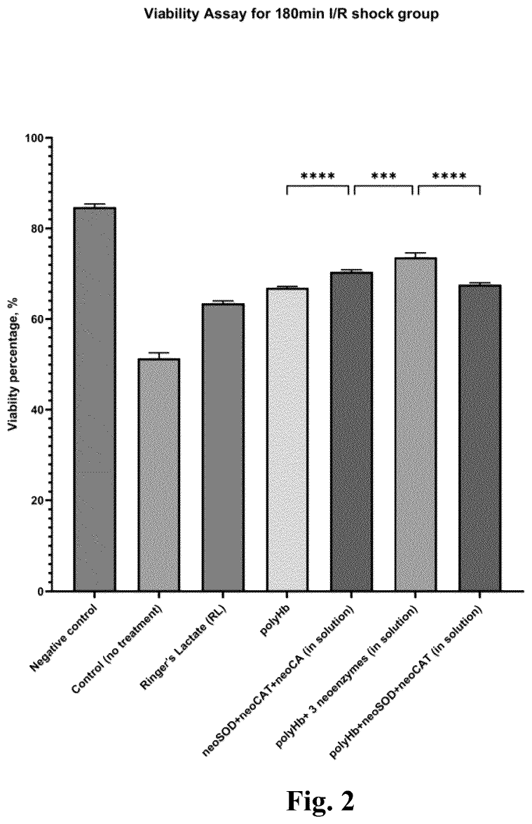

The viability percentage from this preliminary study showed that the polyHb+at least two neoenzymes had high viability percentage and polyHb+the three neoenzymes in solution had a better impact on the cells after a I/R shock as compared to the control and other groups as can be seen in FIG. 2. PolyHb alone could not have a bigger viability percentage, which indicates the production of oxygen radicals that were not removed from the media during the 24 hr incubation. Just neoenzymes had a better viability compared to polyHb, however, that too had limitations as the one with an oxygen carrier i.e., polyHb along with all three neoenzymes showed better viability. The best effect is PolyHb+all three neoenzymes since this supplies oxygen, remove CO2 and oxygen radicals

### Example 3

Cardiomyocyte protective effects of PolyHb+neoCAT+neoSOD+neoCA and PolyHb+neoCAT+neoSOD in solution on an In vitro Ischemic/Reperfusion (I/R) model of human cardiomyocyte

This is also a feasibility study on the use of PolyHb-CAT-SOD-CA for the preservation and recovery of ischemic cells and organs for transplantation. Organs and cells for transplant are usually ischemic during the period they are removed from the diseased patients. This is also to see if it can protect the heart from damage in severe hemorrhagic shock when treated with PolyHb alone. The above fluid supplies the needed oxygen and removes the accumulated CO₂ and oxygen radicals and helps the organs and cells to recover before transplantation. This feasibility is shown in our following cardiomyocyte study of improved viability.

### Methods:

### Human Cardiomyocyte Culture:

PromoCell Myocyte Growth Medium (C-22070, Sigma Aldrich Canada) (supplemented with 5% % FBS and 1% penicillin/streptomycin) was warmed to 37° C. in a water bath. The cryopreserved cardiomyocytes were thawed in 37° C. water bath for <2 min. The vial was then wiped with 70% alcohol in hood and the content was then transferred into the warm PromoCell Myocyte Growth Medium. It was then centrifuged at 100×g for 10 min. The supernatant was poured off into waste bottle and ˜1 ml of the myocyte medium per 1×10⁶ total cells i.e., 8 ml for 8×10⁶ cells was added and mixed gently. Cells were then counted and seeded at a density of 2.4×10⁵ cells/ml on 6 well plates. The plates were marked according to the different groups of study, which were:
cardiomyocytes with no ischemic shock (negative control)
Cardiomyocytes after Ischemic shock with no treatment (control with no treatment).
Cardiomyocytes treated with 20 ul RL after Ischemic shock
Cardiomyocytes treated with 0.626 mmol/L PolyHb after Ischemic shock
Cardiomyocytes treated with 55.6 mmol/L of each of three neoenzymes in solution (neoSOD+neoCAT+neoCA) after Ischemic shock
Cardiomyocytes treated with 0.626 mmol/L PolyHb+55.6 mmol/L of each of three neoenzymes in solution (neoSOD+neoCAT+neoCA) after Ischemic shock
Cardiomyocytes treated with 0.626 mmol/L PolyHb+55.6 mmol/L of each of two neoenzymes in solution (neoSOD+neoCAT) after Ischemic shock

The plates were incubated at 37° C. for 6 hrs. After incubation, the plates were agitated gently to loosen debris and the medium was aspirated and replaced with warm supplemented myocyte medium and kept at incubator for 24 hrs.

### I/R In Vitro Model

Also used as a test for the preservation of ischemic cells and organs for transplantation. Organs and cells for transplant are usually ischemic during the period they are removed from the diseased patients. This is also to see if it can protect the heart from damage in severe hemorrhagic shock when treated with PolyHb The above fluid supplies the needed oxygen and removes the accumulated CO₂ and oxygen radicals and helps the organs and cells to recover before transplantation. This feasibility is shown in our cardiomyocytes study of improved viability.

The plates were taken out of the incubator and were placed in a shaker kept at 37 C along with a constant flow of Nitrogen gas inside the shaker for creating an ischemic model. The cells were kept in that low oxygen environment for 180 minutes. The cells were then taken out of the shaker and the media of the cells were replaced with fresh supplemented myocyte media and the following treatment was given into previously labelled wells:
Control cardiomyocytes got only fresh myocyte media
Cardiomyocytes treated with 20 ul RL
Cardiomyocytes treated with 0.626 mmol/L PolyHb
Cardiomyocytes treated with 55.6 mmol/L of each of three neoenzymes in solution (neoSOD+neoCAT+neoCA)
Cardiomyocytes treated with 0.626 mmol/L PolyHb+55.6 mmol/L of each of three neoenzymes in solution (neoSOD+neoCAT+neoCA)
Cardiomyocytes treated with 0.626 mmol/L PolyHb+55.6 mmol/L of each of two neoenzymes in solution (neoSOD+neoCAT)

The plates were then placed back at the incubator for 24 hours.

### Viability Assay:

The liquid media was aspirated using pipettes. The cells were then washed twice with 2 ml PBS. 1 ml of 4 mg/ml working solution of Trypsin was added onto each well. The plate was gently swirled and keep at RTP for 10 min. 1.5 ml (3:1 of media: trypsin) of myocyte Media (supplemented with FBS) was added to stop collagenase. These were then taken into each separate centrifuge tubes. 1 ml of media was added to the plates and washed and transferred them to the respective tubes. It was repeated 2 more times. The cells were then centrifuged at 1500 rpm for 5 min and the supernatant was then discarded followed by re-suspending the pellet with 5 ml of media. The cells were then counted with trypan blue method using the formula: percentage viability=(total no of cells-total no of cells in blue)/total no of cells×100%.

### Results:

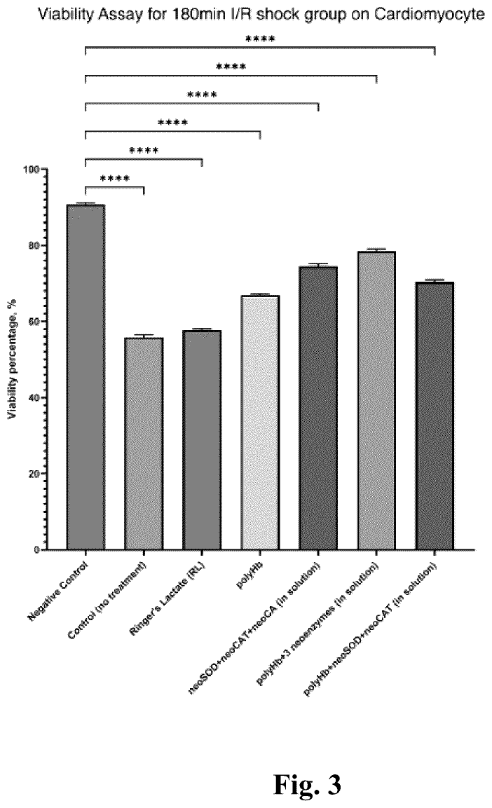

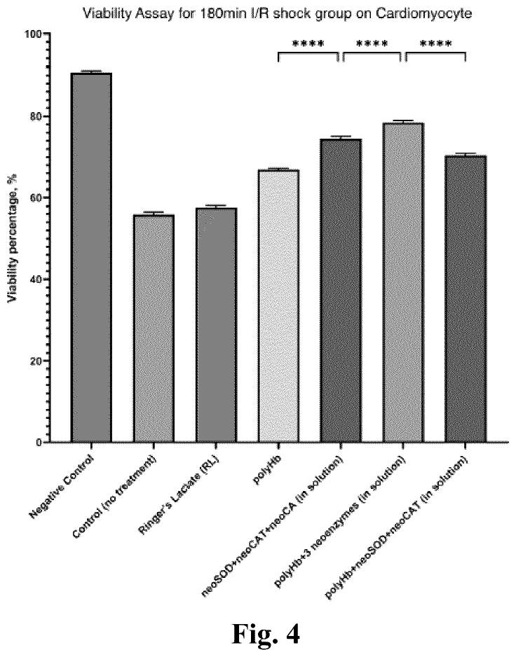

The viability percentage from this preliminary study showed that the polyHb+at least two neoenzymes had high viability percentage and polyHb+the three neoenzymes in solution had a better impact on the cells after a I/R shock as compared to the control and other groups as can be seen in FIG. 3 and FIG. 4. PolyHb alone could not have a bigger viability percentage, which indicates the production of oxygen radicals that were not removed from the media during the 24 hr incubation. Just neoenzymes had a better viability compared to polyHb, however, that too had limitations as the one with an oxygen carrier i.e., polyHb along with neoenzymes showed better viability. The best effect is PolyHb+all three neoenzymes since this supplies oxygen, remove CO₂ and oxygen radicals.

While preferred embodiments have been described above and illustrated in the accompanying drawings, it will be evident to those skilled in the art that modifications may be made without departing from this disclosure. Such modifications are considered as possible variants comprised in the scope of the disclosure.

1. A synthetic enzymatic activity solution comprising a Neo-Carbonic Anhydrase (neoCA) compound having Carbonic Anhydrase activity chosen from the group consisting of ZnHisGly, (Zinc Histidine Glycerol), and at least one compound selected from the group consisting of:
a Neo-Superoxide Dismutase (neoSOD) compound having Superoxide Dismutase activity chosen from the group consisting of MnTmPyP, or Mn(III)tetrakis(1-methyl-4-pyridyl)porphyrin;
a Neo-Catalase (neoCAT) compound having Catalase activity chosen from the group consisting of EUK-134, or 2,2′-[1,2-Ethanediylbis(nitrilomethylidyne)]bis[6-methoxy-phenol manganese complex; Chloro[[2,2′-[1,2-ethanediylbis(nitrilomethylidyne)]bis[6-methoxyphenolato]](2-)-N2,N2′,O1,O1′]-manganese);
wherein the at least one compound and the Neo-Carbonic Anhydrase (neoCA) are in a PBS solution (pH-7.4), saline, Ringer Lactate, cell culture media, organ preservation media, distilled water dH₂O or other pharmaceutically acceptable buffer and is adapted to transport carbon dioxide and/or remove oxygen radicals.

2. An artificial blood substitute solution comprising:
the synthetic enzymatic activity solution of claim 1 having a combination of up to 3 compounds; and
an oxygen carrier selected from the group consisting of: PolyHb, other modified hemoglobin (examples include but not limited to other types of polyhemoglobin, conjugated hemoglobin including PEG-hemoglobin, intramolecularly crosslinked hemoglobin, recombinant hemoglobin, bioengineered hemoglobin, nanoencapsulated hemoglobin with PEG-lipid membrane or PEG-PLA membrane or other membranes), perfluorochemicals, synthetic heme, oxygen saturated solution; fluid for cell and organ preservation used in organs and cell preservation for transplantation or for biotechnological production of stem cells and other cells;
wherein the artificial blood substitute solution is adapted to transport both carbon dioxide and oxygen and to remove oxygen radicals.

3. The artificial blood substitute solution according to claim 2, wherein the ratio of oxygen carrier to the neoCA, neoSOD and neoCAT is oxygen carrier from 0 to 10 g and neoenzymes from 1 mg to 180 mg.

4. The synthetic enzymatic activity solution of claim 1, wherein said synthetic enzymatic activity solution consists of the Neo-Carbonic Anhydrase (neoCA) compound having Carbonic Anhydrase activity, the Neo-Superoxide Dismutase (neoSOD) compound having Superoxide Dismutase activity and the Neo-Catalase (neoCAT) compound having Catalase activity.

5. A method for the treatment of a blood loss, a condition with accumulation of carbon dioxide, an ischemia-reperfusion injury, or a diver's disease in a patient in need thereof, which comprises administering the artificial blood substitute solution according to claim 2.

6. The method according to claim 5, wherein said ischemia-reperfusion injury comprises a severe hemorrhagic shock, a stroke, a myocardial infarction, or combinations thereof.

7. A method for removing carbon dioxide from a fluid, which comprises treating the fluid to the synthetic enzymatic activity solution according to claim 1 for a time sufficient to remove the carbon dioxide therefrom.

8. The method according to claim 7, wherein said fluid is chosen from blood and air.

9. The method according to claim 8, wherein said fluid is blood in an artificial lung.

10. The method according to claim 8, wherein said fluid is blood in a dialysis.

11. The method according to claim 8, wherein said blood in an artificial lung is for transporting oxygen and removing CO₂ in a patient (for example severe COVID patients).

12. A method for preservation of organs or cells for transplantation or for preserving cells like stem cells and other cells for industrial production and storage, which comprises treating the organs or the cells with the artificial blood substitute solution according to claim 2.
